(19) United States
(12) Patent Application Publication
(10) Pub. No.: US 2011/0052648 A1
(43) Pub. Date: Mar. 3, 2011

(54) ORAL MODIFIED-RELEASE FORMULATIONS CONTAINING THIAZEPINES

(76) Inventors: Avi Avramoff, Haifa (IL); Adel Penhasi, Nolon (IL); Orly Einav-Rubashkin, Haifa (IL); Ron Schlinger, Tel-Aviv (IL)

(21) Appl. No.: 12/921,168

(22) PCT Filed: Mar. 9, 2009
(86) PCT No.: PCT/IL2009/000257

§ 371 Date: Nov. 16, 2010

Related U.S. Application Data
(60) Provisional application No. 61/064,556, filed on Mar. 12, 2008.

(51) Int. Cl. A61K 31/554; A61K 9/00; A61P 25/22; A61P 25/18; A61P 25/24

(52) U.S. Cl. 424/400; 514/211.13

(57) ABSTRACT

An oral modified-release formulation using Quetiapine or pharmaceutically acceptable salts thereof as an active ingredient, while avoiding the use of a gelling material. As used herein, the term “modified release” includes but is not limited to one or more of controlled release, sustained release, prolonged release and extended release.

### FIELD OF THE INVENTION

The present invention relates to oral formulations of thiazepines, and in particular, to novel oral formulations which provide modified release.

### BACKGROUND OF THE INVENTION

Thiazepines are substituted thiepins, with a nitrogen replacing a carbon in the seven-membered heterocyclic compound.

One member of the thiazepine family is the dibenzothiazepine, Quetiapine, an atypical antipsychotic which has antidopaminergic activity. The mechanism of action is unknown. However, it is thought that the drug's therapeutic activity in schizophrenia is mediated through a combination of dopamine type 2 (D2) and serotonin type 2 (5HT₂) receptor antagonism. Although Quetiapine is known to bind other receptors with similar affinity, only the dopamine D2 and serotonin 5HT₂ receptor binding is responsible for quetiapine's therapeutic activity in schizophrenia. The preparation, physical properties and beneficial pharmacological properties of this drug, 11-[4-[2-(2-hydroxyethoxy)ethyl]-1-piperazinyl]dibenzo[b,f][1,4]-thiazepine, and its pharmaceutically acceptable salts, are described in published European Patents EP 240,228 and 282,236 as well as in U.S. Pat. No. 4,879,288; additional salts are described in WO 2006/056772 to Pliva Hrskat; the entire contents of all of which are herein incorporated by reference as if fully set forth herein.

Oral formulations of Quetiapine are used for the treatment of Schizophrenia as described in molecule U.S. Pat. No. 4,879,288 (ICI AMERICA INC), the entire contents of which are herein incorporated by reference as if fully set forth herein.

Thiazepine compounds are characterized by limited solubility in water. Quetiapine, for example, is only moderately soluble in water. Modified formulations for thiazepines are therefore difficult to formulate.

Modified release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may be decreased, leading to a more even drug level in the blood over a period of time. Such formulations may also provide a longer plateau concentration of the drug in the blood. The size and frequency of dosing is determined by the pharmacodynamic and pharmacokinetic properties of the drug. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Previous publications (U.S. Pat. No. 5,948,437 to Zeneca Limited) described controlled release formulations of thiazepines such as Quetiapine using gelling agents, especially hydrophilic excipients that create a gel structure after contact with water such as hydroxypropyl methylcellulose and derivatives. Such publications taught of the difficulty of preparing formulations of thiazepines which could be administered once daily while maintaining a suitably high plasma level.

### SUMMARY OF THE INVENTION

The background art does not teach or suggest a suitable formulation for thiazepines such as Quetiapine which is useful for modified release, and which avoids the use of gelling materials.

The present invention provides an oral modified-release formulation using thiazepines or pharmaceutically acceptable salts thereof as an active ingredient, while avoiding the use of a gelling material. As used herein, the term “modified release” includes but is not limited to one or more of controlled release, sustained release, prolonged release and extended release.

The formulation of the present invention preferably comprises one or more non-gelling ingredients, including but not limited to polymethacrylates and their copolymers, polyvinyl acetate, polyvinyl acetate-based copolymers, and any hydrophobic modified cellulose derivatives, non-gelling polysaccharides, non-gelling modified polysaccharides, pharmaceutically acceptable waxes, zein, shellac, copolymers of C₅ to C₃₀ alkyl (meth)acrylates, C₅ to C₃₀ ester of an alkyl (meth)acrylate and the combinations thereof.

The modified release formulation preferably provides a release profile of the drug from the drug product for an extended period of time, for example (and without limitation) several hours of gradual release, thereby enabling once daily administration of the formulation.

The background art fails to teach a suitable non-gelling modified release formulation for thiazepines, preferably dibenzothiazepines and more preferably quetiapine. As previously described, such molecules typically feature low to moderate water solubility, such that the use of gelling materials is less desirable, as they rely upon water to form their gelling structures. The present invention overcomes these disadvantages of the background art by using only non-gelling materials in the formulations of the present invention.

The present invention, in some embodiments, relates to a release mechanism of the API that is not limited to diffusion. Possible release mechanisms under the present invention may optionally include but are not limited to one or more of surface erosion, bulk erosion, dissolution or biodegradation, or the like.

The formulation is optionally in the form of a coated tablet, pellet, granule, microparticle, agglomerate, capsule or any other solid dosage form.

The modified-release solid oral dosage form formulation is preferably manufactured by any suitable well known technique, including but not limited to, wet granulation using high-shear mixer or low-shear mixer, top-spray granulation or a direct compression process, or a multi-layer compression tablet etc. The modified-release agents may be located inside and/or outside the granule and/or as a component of the coating layer covering the tablet core.

The solid oral dose form optionally contains one or more other inactive ingredients, optionally including one or more of diluents, fillers, lubricants, binders, stabilizers, coloring dyes and the like. The formulation may contain one or more of the above ingredients to improve the tablet process, feasibility and release profile.

The combination of the selected materials for the core and outer layer, and the relative concentrations thereof, as well as the thickness of the core matrix and outer layer, collectively determine the rate of release of the drug.

The doses of the active ingredient, preferably Quetiapine, to be used in the formulations of the present invention can be determined by a person of skill in the art, and will vary depending on the active ingredient being used, the patient, and the condition being treated. Typical known therapeutic doses for each of the active ingredients in the thiazepine class can be used as a guide to determine the appropriate dose to be used herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph showing the dissolution profile of an examplary formulation in accordance with the teachings of the present invention, as compared to the reference product (Seroquel, Astra Zeneca).

### DESCRIPTION OF THE PREFERRED EMBODIMENTS

The present invention provides an oral modified-release formulation using a thiazepine or pharmaceutically acceptable salts thereof as an active ingredient, while avoiding the use of a gelling material. Instead, preferably only non-gelling materials are used.

Preferably, the thiazepine comprises either a benzothiapine (including, for example, a 1,4-thiazepine such as Diltiazem, or a 1,3-thiazepine) or a dibenzothiazepine, or a pharmaceutically acceptable salt thereof, or a combination thereof. Non-limiting examples of dibenzothiazepines include quetiapine; clothiapine; 2-(2-(7-hydroxy-4-dibenzo(b,f)(1,4)thiazepine-11-yl-1-piperazinyl)ethoxy)ethanol; 4-methyl-1,2,3,4-tetrahydrodibenzo-1,4-diazepino(2,1-c)-1,4-thiazepine; and metiapine.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

One particularly advantageous example of a suitable dibenzothiazepine is Quetiapine. It should be noted that Quetiapine is described as an example of a therapeutic thiazepine and in particular as an example of a therapeutic dibenzothiazepine, such that the formulations described herein may optionally be extended to therapeutically active compounds of these classes.

Optionally, Quetiapine may be provided as the fumarate salt.

The formulation preferably comprises a core for containing the active ingredient, although optionally the active ingredient may be contained in a coating on a neutral core. The neutral core may comprise, for example, at least one of a non-pareil, a bead, a seed, a granule, or a pellet.

Optionally, the core may further comprise an additional active ingredient, such as, for example, an antipsychotic, antidepressant, or anxiolytic agent.

Examples of suitable antipsychotic agents include typical antipsychotics (such as chlorpromazine, fluphenazine, haloperidol, molindone, thiothixene, thioridazine, trifluoperazine, loxapine, perphenazine, prochlorperazine, pimozide, or zuclopenthixol) or atypical antipsychotics (such as clozapine, risperidone, olanzapine, ziprasidone, aripiprazole, or paliperidone).

Examples of suitable antidepressants include selective serotonin reuptake inhibitors (such as fluoxetine, paroxetine, escitalopram, citalopram, sertraline, and fluvoxamine), serotonin-norepinephrine reuptake inhibitors (such as venlafaxine, milnacipram and duloxetine), noradrenergic and specific serotonergic antidepressants (such as mirtazapine), norephinephrine reuptake inhibitors (such as reboxetine), norepinephrine-dopamine reuptake inhibitors (such as bupropion), and tricyclic antidepressants (such as amitriptyline or desipramine).

Examples of suitable anxiolytic agents include benzodiazepines (such as lorazepam, clonazepam, alprazolam, and diazepam), serotonin 1A agonists (such as buspirone), barbiturates, and hydroxyzine.

The core preferably comprises a filler. Preferably, the filler is selected from the group consisting of microcrystalline cellulose, sodium carboxymethycellulose, ethylcellulose, cellulose acetate, starch (such as corn starch or potato starch), a hydrogenated starch hydrolysate, a sugar (such as lactitol, lactose, glucose, fructose or sucrose), a sugar alcohol (such as sorbitol, manitol, mantitol, lactitol, xylitol, isomalt, or erythritol) a suitable inorganic calcium salt (such as dicalcium phosphate), or a combination thereof. More preferably the filler comprises starch and lactose, such as lactose monohydrate for example. Optionally and preferably, the filler comprises microcrystalline cellulose. The core also preferably comprises a lubricant. Preferably the lubricant is selected from the group consisting of Silica Colloidal Anhydrous and magnesium stearate, or a combination thereof.

The core also preferably comprises a water insoluble polymer. Preferably the water insoluble polymer is selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”; an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”; a dimethylaminoethylmethacrylate/methylmethacrylate and butylmethacrylate copolymer; a copolymer based on neutral methacrylic acid esters and dimethylaminoethyl methacrylate esters wherein the polymer is cationic in the presence of acids; an ethylacrylate and methylacrylate/ethylmethacrylate and methyl methylacrylate copolymer, the copolymer being a neutral copolymer based on neutral methacrylic acid and acrylic acid esters, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

The core, optionally additionally or alternatively, comprises a pH dependent polymer. Preferably, the pH dependent polymer is selected from the group consisting of a hydroxypropylmethyl cellulose phthalate, polyvinyl acetate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate)1:1, alginic acid, and sodium alginate, or combinations thereof.

The core, optionally additionally or alternatively, comprises a water soluble polymer. Optionally and preferably the water soluble polymer comprises polyvinyl alcohol, polyvinylpyrrolidone (PVP), copolyvidone, methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

Optionally and preferably one or more coatings comprising a non-gelling polymer are layered on the core.

More preferably the outer coating comprises a water insoluble polymer. Preferably the water insoluble polymer is selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, shellac, zein, and waxes, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

Optionally and preferably, the copolymer of the outer coating is a pH-dependent copolymer. Preferably, the pH dependent polymer is selected from the group consisting of a hydroxypropylmethyl cellulose phthalate, polyvinyl acetate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate, or combinations thereof.

The coating also optionally and preferably comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

The coating also optionally and preferably comprises a glidant. More preferably the glidant includes at least one of microcrystalline cellulose, talc, colloidal hydrated aluminum silicate (bentonit), or colloidal silicon dioxide (aerosil) or a combination thereof.

According to some embodiments, the formulation of the present invention comprises a tablet comprising an immediate release core, and at least one coating comprising a non-gelling polymer, which is optionally pH dependent. Optionally, the formulation may comprise an outer coating comprising a pH-dependent copolymer, and an intermediate coating layer between the core and the outer coating layer, wherein the intermediate coating layer comprises a non-pH-dependent rate-controlling copolymer.

The coating may optionally separately comprise active material, which may be the same as or different from the active material of the core. Examples of suitable additional active ingredients include any of those described above as optional additional active ingredients for the core.

The below Examples provide some non-limiting, illustrative formulations of the present invention according to some embodiments, as well as methods of manufacture and release profiles thereof.

The formulation of the present invention preferably releases the active ingredient in a controlled fashion, over a period of at least about 4 hours or longer, preferably over a period of at least about 8 hours or longer and in particular over a period of from about 8 to about 24 hours, such that at least about 60% of the active ingredient has been released at the end of this period.

In other embodiments, the formulation preferably releases the active ingredient in a controlled manner over a period of up to about 8 hours or longer. For example, the formulation, in such embodiments, is preferably capable of releasing about 90% of the active ingredient over a period of about 8-16 hours.

Compositions of the present invention may, if desired, be presented in a pack or dispenser device, such as an FDA approved kit, which may contain one or more dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non limiting fashion.

### Example 1

### Slow Release

Quetiapine or a pharmaceutically acceptable salt thereof (API), Lactose, Corn Starch and Magnesium Stearate are mixed together in high-shear mixer. Ammonium Methacrylate copolymer B is added and Acetone is poured into the granulator to form small granulates which are then preferably dried and milled before being compressed to tablets.

The drug is released gradually, such that after 4 to 7 hours, about 80% of the API is released. Optionally, the formulation may be adjusted such that after 8 to 12 hours, more than 80% is dissolved in vitro (dissolution test).

### Example 2

### Slow Release

Quetiapine or salts thereof, polyvinyl acetate, microcrystalline cellulose, silica colloidal anhydrous and magnesium stearate are used.

The polyvinyl acetate is dissolved in adequate amount of acetone and the solution is sprayed onto a mixture of all ingredients (except magnesium stearate) in a granulator. After drying the granules, magnesium stearate is added for final mixing and the resultant mixture is compressed into tablets.

The dissolution of the formulation described in this example may optionally feature the same rates as presented in the two embodiments indicated for Example 1.

The active ingredient is released gradually, such that after 4 to 7 hours, about 80% of the API is released. Optionally, the formulation may be adjusted such that after 8 to 12 hours, more than 80% is dissolved in vitro (dissolution test).

### Example 3

### Fast Disintegrating Tablets with Controlled-Release Coating

Tablet cores are manufactured according to a granulation process in a low-shear mixer with the following formulation (all percentages are weight/weight over the weight of the total formulation):

### Quetiapine Fumarate (API)—20-90%

### Microcrystalline Cellulose—10-70%

### Colloidal Silicon Dioxide—0.5-1.5%

### Magnesium Stearate—0.5-2%

The above ranges (and any other ranges for ingredient amounts in this example or below in other examples) indicate that each of the above ingredients may optionally vary within that range, although all weight/weight percentages must together add up to 100%.

The following preparation process is performed. The API is mixed with microcrystalline cellulose in a low shear mixer. One or more organic solvents for example including but not limited to acetone, ethanol and the like, are sprayed on the mixture and granulate. The wet mass is dried and milled for homogenous particle size distribution.

Colloidal silicon dioxide and magnesium stearate are added to perform the final blend for compression in a tablet punching machine with suitable punches.

### Coating Layer:

Ammonium Methacrylate copolymer A 30%—0-20% of dried polymer
Triethyl Citrate—10-30% of polymer substance
Talc—30-60% of polymer substance

A coating solution is prepared with the above ingredients in purified water and sprayed on the tablet cores in a coating pan.

### Example 4

### Fast Disintegrating Tablets with Controlled-Release Coating

Tablet cores were manufactured according to a granulation process in a low-shear mixer with the following formulation (all percentages are weight/weight of the total formulation):

### Quetiapine Fumarate (API)—20-90%

### Microcrystalline Cellulose—30-50%

### Lactose—30-50%

### Magnesium Stearate—0.5-2%

Core tablets are manufactured as for Example 3, but in this Example the solvent for granulating the mixture is purified water.

### Coating Layer:

Polyacrylate Dispersion 30%—10-30% of dried polymer
Talc—50-100% of polymer substance

After mixing the polymer dispersion with a sufficient amount of glidant, such as talc, in purified water as the solvent, the dispersion is sprayed on the tablet cores in a coating pan.

### Example 5

### Fast Disintegrating Tablets with Controlled-Release Coating

Tablet cores are manufactured according to a granulation process in a low-shear mixer with the following formulation (all percentages are weight/weight over the weight of the total formulation):

### Quetiapine (API)—20-90%

### Sodium Starch Glycolate—2-6%

### Lactose—40-70%

### Povidone—2-10%

### Microcrystalline Cellulose—10-20%

Mg. Stearate—0.5-2%

The granulation process is performed by spraying granulation solution containing povidone and ethanol on the dried mixture. After drying, the granulate is milled and then mixed with microcrystalline cellulose and magnesium stearate before compression of the granulate to tablets.

The tablet cores are coated with a dispersion of a methacrylate co-polymer, such as ammonium methacrylate colpolymer B for example, optionally in an amount of about 30%, with sufficient amounts of talc (glidant) and triethyl citrate (plasticizer).

### Example 6

### Fast Disintegrating Tablets with Controlled-Release Coating

Tablet cores are manufactured according to a granulation process in a low-shear mixer with the following formulation (all percentages are weight/weight over the weight of the total formulation):

Tablet cores are manufactured by direct compression process:

### Quetiapine Fumarate—20-90%

### Lactose—10-70%

### Corn Starch—2-10%

### Colloidal Silicon Dioxide—0.5-2%

### Magnesium Stearate—0.5-2%

Tablet cores are manufactured by mixing all ingredients and compressed into tablets using a punch machine with the appropriate punches.

### Coating Layer:

Ammonium Methacrylate copolymer A 30%—5-20% of dried polymer
Ammonium Methacrylate copolymer B 30%—5-20% of dried polymer
Triethyl Citrate—10-20% of polymer substance
Talc—30-60% of polymer substance

A coating solution is prepared by dissolving the above ingredients in purified water, and then sprayed on the tablet cores in a coating pan.

### Example 7

### Fast Disintegrating Tablets with Controlled-Release Coating

Tablet cores are manufactured according to a granulation process in a high-shear mixer with the following formulation (all percentages are weight/weight over the weight of the total formulation):

### Quetiapine Fumarate—20-90%

### Lactose—10-70%

### PVP—2-5%

### Colloidal Silicon Dioxide—0.5-1.5%

### Magnesium Stearate—0.5-3%

The API is mixed with lactose in high-shear mixer for few minutes. Sufficient amounts of ethanol and PVP are placed in the high-shear mixer bowl and mixed to granulate the material. The wet mass is dried in a fluid bed and milled if necessary. Colloidal silicon dioxide and magnesium stearate are added to perform the final blend before compression.

### Coating Layer:

Solution of Polyvinyl Acetate in organic solvent is sprayed on the tablet to achieve the desired dissolution profile.

### Example 8

### pH-Dependent Coating

A core tablet containing one or more non-gelling agents (optionally pH dependent polymer) is coated with a pH-dependent polymer, for example methacrylic acid copolymer. Tablet cores are manufactured according to a granulation process in a high-shear mixer with the following formulation:

### Quetiapine Fumarate—43.2%

### Avicel PH 101—38.8%

### Eudragit NE-30D—15.0%

### Colloidal Silicon Dioxide—1.0%

### Magnesium Stearate—2.0%

Coating Layer is sprayed onto the core tablet up to a weight gain of 3% (relative to the core tablet weight), the coating consists of the following formulation:

### Metacrylic Acid Copolymer—64.1%

Lactose monohydrate—16.0%

### Talc—13.5%

### Propylene Glycol US—6.4%

The dissolution profile of tablets manufactured according to Example 8 were tested against reference tablets, Seroquel XR 300 mg Tablets, manufactured by Astra Zeneca UK Limited, Macclesfield, UK.

The following dissolution method was used: 750 ml of 0.1 N HCl for 2 hours at 37° C. using paddles (Apparatus-2) at a speed rate of 100 rpm and then adding 250 ml of 0.2M sodium phosphate buffer to the dissolution media to obtain a pH of 6.2.

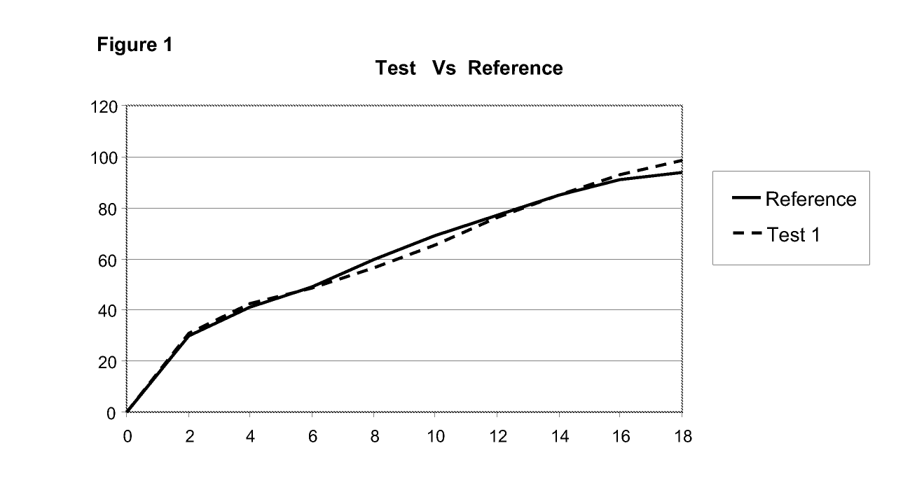

As shown in FIG. 1, the dissolution profile of the test tablet was substantially identical to that of the reference product.

### Example 9

### Immediate Release Core with Controlled Release Coating

An immediate release core with two coatings containing a non-gelling polymer, wherein the first layer contains an ammonium methacrylate copolymer and the second layer contains a pH-dependent methacrylic acid copolymer. Drug release is retarded at low pH by the methacrylic acid copolymer, and once the pH is elevated the second layer dissolves and the release rate is controlled by the first layer containing ammonium methacrylate copolymer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. (canceled)

2. An oral modified-release formulation comprising a thiazepine compound or a pharmaceutically acceptable salt thereof and at least one polymer selected from the group consisting of a polymethacrylate, a polymethacrylate copolymer, polyvinyl acetate, a polyvinyl acetate-based copolymer, a hydrophobic modified cellulose derivative, a non-gelling polysaccharide, a non-gelling modified polysaccharide, zein, shellac, a copolymer of C₅ to C₃₀ alkyl (meth)acrylates, a C₅ to ₃₀ ester of an alkyl (meth)acrylate and combinations thereof, wherein said formulation is devoid of a gelling agent.

3. The formulation of claim 2, wherein the thiazepine compound is selected from the group consisting of a benzothiapine or a dibenzothiazepine, or a pharmaceutically acceptable salt thereof.

4. The formulation of claim 2, wherein the thiazepine compound comprises a dibenzothiazepine selected from the group consisting of quetiapine; clothiapine; 2-(2-(7-hydroxy-4-dibenzo(b,f)(1,4)thiazepine-11-yl-1-piperazinyl)ethoxy)ethanol; 4-methyl-1,2,3,4-tetrahydrodibenzo-1,4-diazepino(2,1-c)-1,4-thiazepine; and metiapine.

5. The formulation of claim 4, wherein said dibenzothiazepine comprises Quetiapine or a pharmaceutically acceptable salt thereof.

6. The formulation of claim 5, wherein said pharmaceutically acceptable salt of quetiapine is selected from the group consisting of a sulfate, hydrochloride, phosphate, sulfamate, acetate, citrate, lactate, tartrate, methanesulfonate, ethanesulfonate, benzenesulfonate, p-toluenesulfonate, cyclohexylsulfamate, fumarate and quinate salt.

7. The formulation of claim 6, wherein said quetiapine salt comprises quetiapine fumarate.

8. The formulation of claim 2, further comprising a filler.

9. The formulation of claim 8, wherein said filler is selected from the group consisting of microcrystalline cellulose, starch, a sugar, a sugar alcohol, a suitable inorganic calcium salt, sucrose, or a combination thereof.

10. The formulation of claim 9, wherein said filler comprises starch and lactose.

11. The formulation of claim 9, wherein said filler comprises microcrystalline cellulose.

12. The formulation of claim 2 further comprising a lubricant.

13. The formulation of claim 12, wherein said lubricant is selected from the group consisting of Silica Colloidal Anhydrous and magnesium stearate, or a combination thereof.

14. The formulation of claim 2, further comprising a water insoluble polymer.

15. The formulation of claim 14, wherein said water insoluble polymer is selected from the group consisting of a podimethylaminoethylacrylate/ethylmethacrylate copolymer, a ethylmethacrylate/chlorotrimethylammoniumethyl methacrylate copolymer, a dimethylaminoethylmethacrylate/methylmethacrylate and butylmethacrylate copolymer; a copolymer based on neutral methacrylic acid esters and dimethylaminoethyl methacrylate esters; an ethylacrylate and methylacrylate/ethylmethacrylate and methyl methylacrylate copolymer; shellac, zein, a wax, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

16. The formulation of claim 2, further comprising a pH dependent polymer.

17. The formulation of claim 16, wherein said pH dependent polymer is selected from the group consisting of hydroxypropylmethyl cellulose phthalate, polyvinyl acetate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate)1:1, alginic acid, and sodium alginate, or a combination thereof.

18. The formulation of claim 2, further comprising a water soluble polymer.

19. The formulation of claim 18, wherein said water soluble polymer is selected from the group consisting of polyvinyl alcohol, polyvinylpyrrolidone (PVP), copolyvidone, methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and mixtures thereof.

20. The formulation of claim 2, further comprising at least one coating layer.

21. The formulation of claim 20, wherein said coating comprises an active ingredient.

22. The formulation of claim 21, wherein said coating layer comprises a water insoluble polymer.

23. The formulation of claim 22, wherein said water insoluble polymer is selected from the group consisting of a podimethylaminoethylacrylate/ethylmethacrylate copolymer, a ethylmethacrylate/chlorotrimethylammoniumethyl methacrylate copolymer, a dimethylaminoethylmethacrylate/methylmethacrylate and butylmethacrylate copolymer; a copolymer based on neutral methacrylic acid esters and dimethylaminoethyl methacrylate esters; an ethylacrylate and methylacrylate/ethylmethacrylate and methyl methylacrylate copolymer; shellac, zein, a wax, paraffin, cellulose acetate, cellulose propionate, cellulose acetate propionate, cellulose acetate butyrate, cellulose acetate phthalate, cellulose triacetate, poly (methyl methacrylate), poly(ethylmethacrylate), poly (butyl methacrylate), poly (isobutyl methacrylate), and poly (hexyl methacrylate), poly (isodecyl methacrylate), poly(lauryl methacrylate), poly (phenyl methacrylate), poly (methylacrylate), poly (isopropyl acrylate), poly (isobutyl acrylate) poly(octadecyl acrylate), poly (ethylene), poly (ethylene) low density, poly(ethylene) high density, poly (ethylene oxide), poly (ethyleneterephthalate), poly (vinyl isobutyl ether), poly (vinyl acetate), poly(vinyl chloride) and polyurethane, and/or mixtures thereof.

24. The formulation of claim 20, wherein said coating layer further comprises a pH dependent polymer.

25. The formulation of claim 24, wherein said pH dependent polymer is selected from the group consisting of hydroxypropylmethyl cellulose phthalate, polyvinyl acetate, polyvinyl acetate phthalate, cellulose acetate phthalate, hydroxypropylmethyl cellulose acetate succinate, poly(methacrylic acid, methyl methacrylate) 1:1 and poly(methacrylic acid, ethyl acrylate)1:1, alginic acid, and sodium alginate, or a combination thereof.

26. The formulation of claim 20, wherein said coating layer further comprises a plasticizer.

27. The formulation of claim 26, wherein said plasticizer is selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

28. The formulation of claim 20, further comprising a glidant.

29. The formulation of claim 28, wherein said glidant is selected from the group consisting of microcrystalline cellulose, talc, colloidal hydrated aluminum silicate (bentonit), or colloidal silicon dioxide (aerosil) or a combination thereof.

30. The formulation of claim 20, further comprising an intermediate layer beneath said coating layer.

31. The formulation of claim 2, wherein said thiazepine is released over a period of at least 4 hours.

32. The formulation of claim 2, comprising an active core comprising said thiazepine.

33. The formulation of claim 2, comprising a neutral core and an active layer comprising said thiazepine.

34. The formulation of claim 2 further comprising an additional active ingredient.

35. The formulation of claim 34, wherein said additional active ingredient is selected from the group consisting of an antipsychotic, antidepressant, and an anxiolytic agent.

36. An oral modified release formulation comprising Quetiapine or a pharmaceutically acceptable salt thereof, lactose, corn starch, magnesium stearate, ammonium methacrylate copolymer B and acetone.

37. An oral modified release formulation comprising Quetiapine or a pharmaceutically acceptable salt thereof, polyvinyl acetate, microcrystalline cellulose, silica colloidal anhydrous, and magnesium stearate.

38. (canceled)

39. The oral modified release formulation of claim 20, wherein said outer coating layer comprises a pH-dependent co-polymer.

40. The oral modified release formulation of claim 39, wherein said pH-dependent co-polymer comprises methacrylic acid copolymer.

41. The oral modified release formulation of claim 30, wherein said intermediate layer comprises a non-pH-dependent rate-controlling polymer.

42. The oral modified release formulation of claim 41, wherein said non-pH-dependent rate-controlling polymer comprises ammonium methacrylate copolymer.

43. The formulation of claim 24, wherein said coating layer further comprises lactose monohydrate.
